(19) United States
(12) Patent Application Publication
(10) Pub. No.: US 2016/0223525 A1
(43) Pub. Date: Aug. 4, 2016

(54) METHOD OF SCREENING FOR COMPOUNDS USEFUL IN THE TREATMENT OF ALZHEIMER DISEASE

(71) Applicant: INNOVATIVE CONCEPTS IN DRUG DEVELOPMENT, Gemenos (FR)

(72) Inventors: NATHALIE COMPAGNONE, AURIOL (FR); CLOTILDE BISCARRAT, LA CADIERE D'AZUR (FR)

(21) Appl. No.: 15/021,967

(22) PCT Filed: Sep. 22, 2014
(86) PCT No.: PCT/EP2014/070138

§ 371 Date: Mar. 15, 2016

(30) Foreign Application Priority Data
Sep. 20, 2013 (EP) 13306289.3

(51) Int. Cl. G01N 33/50

(52) U.S. Cl. CPC G01N 33/5079; G01N 33/5091; G01N 33/502; G01N 2800/52; G01N 2800/2821

(57) ABSTRACT

The present invention relates to a method of screening for compounds useful in the treatment of Alzheimer disease using a signature based on mitochondrial behaviour variables. The invention also relates to a method for diagnosing Alzheimer disease and a method for monitoring the response of a subject affected with Alzheimer disease to therapy.

### FIELD OF THE INVENTION

The present invention relates to the field of medicine, in particular to the diagnosis and treatment of Alzheimer's disease.

### BACKGROUND OF THE INVENTION

Alzheimer disease is the most common form of progressive dementia in the elderly. It is a neurodegenerative disorder characterized by the neuropathologic findings of intracellular neurofibrillary tangles and extracellular amyloid plaques that accumulate in vulnerable brain regions. Because Alzheimer's disease represents a prevalence of 15.4% in people over 65 years old, it is a terrible health and societal burden. Furthermore, due to the ageing of the population, the prevalence of AD in Europe is projected to reach 25% in people over 65 by 2025, and to amount to 100 million people worldwide by 2050.

Drug development in Alzheimer's disease (AD) has raised a lot of hope but is currently faced by a very high-rate of failures in late stage clinical trials. The reasons for clinical trial discontinuation have mostly lied upon unrecognized toxicity and lack of demonstration of treatment efficacy. Treatments in AD may aim at the attenuation of cognitive symptoms and/or at the retardation of disease progression. In particular, considering that amyloid deposits and neurofibrillary tangles appear decades before telltale symptoms, it is suggested that it may be possible to design “disease-modifying” drugs, i.e. drugs that target the earliest biological changes in AD to delay symptom onset and disability. Indeed, delaying the onset of disease progression by only 3.5 years will reduce the prevalence of the disease by one third and treating the disease to further delay its progression by 2.8 years will eliminate the need for institutionalization of AD patients.

Furthermore, it is important to appropriately diagnose the disease at stages where treatments have the highest chance of success. However, diagnosis of AD is long and costly as it is mostly based on neurological examination of patient's decline other a period of time. The typology of cognitive decline associated to AD is not a sensitive marker as several loss of functions seen in AD are also present in other dementia. Imaging can provide clinical support in establishing the diagnosis of dementia as it identified regional cell loss but has also low specificity with regards to AD compared with other dementia.

Large-scale analyses of the genome, the transcriptome, the proteome or the metabolome have yielded to the discovery of various markers associated to clinical forms of AD. For example, markers known to be associated to AD include genetic determinants such as mutations of the presenilin gene responsible for 5% of the genetic cases of AD or the presence of the APOE4 allele. Circulating levels of amyloid beta peptides, particularly the Ab40 and Ab42 forms, are also associated with early disease progression and increased ab40/42 ratio are seen in prodromal forms of the disease recognized as mild cognitive impairment (MCI). Furthermore, the presence of high levels of phosphorylated forms of the Tau protein compared to the total Tau in the cerebrospinal fluid is also considered as a converging marker of AD diagnosis. Various other markers have been identified but they are only slowly entering the clinical practice because of lack of validation of their clinical value or of conflicting results are found in the literature.

Unfortunately, dementia is not an easy condition for either families or physicians to deal with. Detection of dementia due to AD and other causes in the primary care setting usually occurs 2 to 4 years after symptom onset, if at all. A recent study found that 75% of moderate and severe dementia cases and >95% of mild dementia cases are not detected in the primary care setting.

Furthermore, even when dementia is detected, about 75% of physicians do not use the standardized criteria needed to accurately diagnose AD.

Therefore, there is a great need of methods of selecting drugs useful in the treatment of AD, in particular disease-modifying drugs, and methods for diagnosing AD and selecting patients for early therapeutic intervention.

### SUMMARY OF THE INVENTION

Using molecular imaging of mitochondria in human live cells, the inventors identified a set of markers that is specific to AD and can be used to diagnose AD at early or asymptomatic stage of the disease. Furthermore, they demonstrated that the signature obtained with these markers can be reversed by treatments and thus provides a human-derived model to screen drugs capable of reversing disease progression of AD.

Accordingly, in a first aspect, the present invention relates to a method, preferably an in vitro method, of screening for compounds useful in the treatment of Alzheimer disease, wherein the method comprises

a) contacting said living cells obtained from a sample from a subject affected with Alzheimer disease with a test compound; and

b) measuring in said contacted cells, the values of the mitochondrial behaviour variables (i) to (iii):

(i) the average cell area, or a dispersion descriptor of the areas of cells (V5);

(ii) a variable selected from the group consisting of the frequency of mitochondria displaying an area between 70 and 100 μm² (V29), the total area of regions containing entwined mitochondria to the total cell area (V7), and the frequency of mitochondria displaying an area between 100 and 200 μm² (V30), and any combination thereof; and

(iii) the average cell area covered with mitochondria expressed as a percent of total cell area, or a dispersion descriptor of the areas of cells covered with mitochondria (V11), and, optionally, the values of the mitochondrial behaviour variables (iv) and/or (v):

(iv) a dispersion descriptor of the moving speeds of mitochondria (V3), and

(v) the frequency of mitochondria displaying an area between 2.6 and 3.1 μm² (V19).

The method may further comprise comparing the values obtained in step b) with the values obtained in absence of the test compound.

The method may further comprise calculating a score for each variable using the following equation:

score=(NC−Var)*100/(NC−PC),

wherein NC is the value or average value obtained with the sample(s) from subject(s) affected with Alzheimer disease in absence of the test compound, PC is the value or average value obtained with healthy sample(s), and Var is the measured value of the variable. A test compound may be identified as useful in the treatment of Alzheimer disease when all measured variables have a positive score.

In a second aspect, the present invention also relates to a method, preferably an in vitro method, for diagnosing Alzheimer disease in a subject, wherein the method comprises:

a) measuring in living cells obtained from a sample from said subject the values of the mitochondrial behaviour variables (i) to (iii):

(i) the average cell area, or a dispersion descriptor of the areas of cells (V5);

(ii) a variable selected from the group consisting of the frequency of mitochondria displaying an area between 70 and 100 μm² (V29), the total area of regions containing entwined mitochondria to the total cell area (V7), and the frequency of mitochondria displaying an area between 100 and 200 μm² (V30), and any combination thereof; and

(iii) the average cell area covered with mitochondria expressed as a percent of total cell area, or a dispersion descriptor of the areas of cells covered with mitochondria (V11),

and, optionally, the values of the mitochondrial behaviour variables (iv) and/or (v):

(iv) a dispersion descriptor of the moving speeds of mitochondria (V3), and

(v) the frequency of mitochondria displaying an area between 2.6 and 3.1 μm² (V19).

The method may further comprise determining if said subject is affected with AD based on the measured values of mitochondrial behaviour variables.

Preferably, the subject is a pre-symptomatic Alzheimer disease patient.

Preferably, the subject has clinical signs that resemble Alzheimer disease or is without any symptom.

The method may further comprise calculating the z-scores of measured variables. Preferably, positive z-scores of the 2^{nd} variable, preferably V7, and the variable V11 and negative z-score of variable V5, are indicative that the subject is affected with Alzheimer disease. Alternatively, positive z-scores of the 2^{nd} variable, preferably V7, V11 and V19 and/or V3, and negative z-score of variable V5, are indicative that the subject is affected with Alzheimer disease at the asymptomatic stage.

In a third aspect, the present invention further relates to a method, preferably an in vitro method, for monitoring the response of a subject affected with Alzheimer disease to therapy, wherein the method comprises

a) measuring in living cells obtained from a sample from said subject, before and after the administration of the treatment, the values of the mitochondrial behaviour variables (i) to (iii):

(i) the average cell area, or a dispersion descriptor of the areas of cells (V5);

(ii) a variable selected from the group consisting of the frequency of mitochondria displaying an area between 70 and 100 μm² (V29), the total area of regions containing entwined mitochondria to the total cell area (V7), and the frequency of mitochondria displaying an area between 100 and 200 μm² (V30), and any combination thereof; and

(iii) the average cell area covered with mitochondria expressed as a percent of total cell area, or a dispersion descriptor of the areas of cells covered with mitochondria (V11),

and, optionally, the values of the mitochondrial behaviour variables (iv) and/or (v):

(iv) a dispersion descriptor of the moving speeds of mitochondria (V3), and

(v) the frequency of mitochondria displaying an area between 2.6 and 3.1 μm² (V19); and

b) comparing the measured values obtained before and after the administration of the treatment in step a).

The method may further comprise calculating a score for each variable using the following equation:

score=(NC−Var)*100/(NC−PC),

wherein NC is the value obtained before the administration of the treatment, PC is the value or average value obtained with healthy sample(s), and Var is the measured value of the variable. Preferably, the patient is responsive to the therapy or is susceptible to benefit from the therapy when all measured variables have a positive score.

In particular, these methods may comprise measuring the mitochondrial behaviour variables V3, V5, V7, V11 and V19. They may also further comprises measuring at least one additional mitochondrial behaviour variable selected from the group consisting of variables (vi) and (vii):

(vi) the average number of individual mitochondria per unit of cell area, or a dispersion descriptor of the numbers of individual mitochondria per unit of cell area (V12); and

(vii) a variable selected from the group consisting of the average frequency of stops during trajectories of individual mitochondria (V1) and the average frequency of burst during displacement of individual mitochondria (V34), and a combination thereof.

These methods may further comprises comparing the measured values of mitochondrial behaviour variables with the values of said variables measured in a sample obtained from a healthy subject.

Preferably the sample is selected from the group consisting of skin biopsy sample, nervous tissue biopsy sample and serum or blood sample. More preferably, the sample is skin biopsy sample.

The cells may be selected from the group consisting of fibroblasts, induced pluripotent stem cells derived from fibroblasts, lymphocytes and neuronal cells. Preferably, the cells are fibroblasts.

Preferably, the dispersion descriptor is selected from the group consisting of the variance, the standard deviation and an interquantile range.

Preferably, before measuring the values of the mitochondrial behaviour variables, mitochondria contained in said living cells are labelled. Mitochondria may be labelled using any suitable label, preferably using a fluorescent label, and more preferably using MitoTracker Green.

The values of mitochondrial behaviour variables may be obtained from images captured using a fluorescence microscope or a differential interference-contrast (DIC) microscope coupled to a suitable image acquisition device, preferably a CCD camera. Preferably, the values of variables are obtained from images captured using a fluorescence microscope coupled to a CCD camera.

### BRIEF DESCRIPTION OF THE DRAWINGS

FIG. 1: Dendogram of the hierarchical cluster analysis applying the Ward's method.

FIG. 2: Heat map representation of the classification of the different subjects in the three subgroups identified by hierarchical clustering. The graph shows the z-scores values for the five variables constituting the AD-signature. The patient labelled “O” is a patient genetically at risk of AD for which no information was available as to the progression of the disease. Our classification implies that the disease was present at the time of the biopsy even if not clinically expressed.

FIG. 3: AD and PAD-signatures of the invention shown by the z-score of each variable in healthy and diseased subject groups.

FIG. 4: Dose-response analysis of Resveratrol of the 5 different variables (V3, V5, V7, V11 and V19) of the AD-signature of the invention (% of phenotypic rescue).

FIG. 5: Weighted representation of the dose-response effect of Resveratrol on disease-mo dification.

FIG. 6: Dose-response analysis of allopregnanolone on the 5 different variables (V3, V5, V7, V11 and V19) of the AD-signature of the invention (% of phenotypic rescue).

FIG. 7: Weighted representation of the dose-response effect of allopregnanolone on disease-modification.

FIG. 8: Dose-response analysis of imatinib on the 5 different variables (V3, V5, V7, V11 and V19) of the AD-signature of the invention (% of phenotypic rescue).

FIG. 9: Weighted representation of the dose-response effect of imatinib on disease-modification.

### DETAILED DESCRIPTION OF THE INVENTION

The inventors previously described that analyzing the behaviour of an organelle, in particular mitochondrion, in a live cell can yield significant information to predict the effect of a compound on an animal or human organism. They defined said behaviour by analyzing the membrane permeability, the dynamic motility of the organelle inside the live cell through space and time, the dynamic changes occurring in the organelle morphology, and the interaction of the organelle with its cellular environment such as the dynamic relationship existing between organelle and elements of the cytoskeleton. This general strategy was disclosed in the U.S. Pat. No. 8,497,089, the content of which is herein enclosed in its entirety by reference. The inventors thus herein consider the global functionalities of the organelle and not solely the individual functions in cellular metabolism.

Because mitochondria are sensitive to the cellular environment and constantly communicate with this environment, they generate thousands of protein-protein interactions within the mitochondria and with other cellular organelles and compartments. Using molecular imaging of mitochondria in live cells, the inventors experimentally and dynamically assessed the mitochondrial reticular system in live cells allowing the capture of the resultant of those interactions that describe the mitochondrial behaviour. They measured 37 mitochondrial behaviour variables relating to the motility, the morphology, the network organization and the permeability of the mitochondria. Motility variables comprise, for example, measures of speed of displacement, amplitude, frequency and regularity of movements as well as distance traveled. Morphology variables comprise, for example, measures of mitochondrial dynamics (fusion-fission balance) and the frequency of apparition of various typical morphological features. The mitochondrial reticular network organization is scored with respect to its orientation, distribution and regionalization with respect to the intracellular cytoskeleton and/or to particular hot-spots in the cells such as the microtubule organizing center and focal adhesion points. Mitochondrial membrane permeability is measured by the dynamic analysis of signal intensity within individual mitochondria.

Based on this analysis, the inventors identified an AD-signature comprising only three mitochondrial behaviour variables that are sufficient to segregate healthy and AD patients. They further demonstrated that this signature can be pharmacologically reversed and can thus be used to screen compounds useful in the treatment of AD.

### DEFINITIONS

As used herein, the term “subject” or “patient” refers to an animal, preferably to a mammal, even more preferably to a human.

As used herein, the term “AD patient” or “AD subject” refers to a subject who is affected with Alzheimer disease. The subject may be at asymptomatic or symptomatic stage of the disease.

The term “Pre-symptomatic Alzheimer disease patient”, “PAD patient” or “PAD subject” refers to a subject who is affected with Alzheimer disease but at the asymptomatic stage of the disease.

As used herein, the term “healthy patient” or “healthy subject” refers to a subject who is not affected with AD. Preferably, the subject is not affected with any known disease, i.e. apparently healthy.

As used herein, the term “treatment”, “treat” or “treating” refers to any act intended to ameliorate the health status of patients such as therapy, prevention and retardation of AD. In certain embodiments, such term refers to the amelioration or eradication of AD or symptoms associated with AD, in particular neurological or cognitive symptoms. In other embodiments, this term refers to minimizing the worsening of AD resulting from the administration of one or more therapeutic agents to a subject affected with AD.

As used herein, the term “sample” means any sample containing living cells derived from a subject. Examples of such samples include fluids such as blood, plasma, saliva, urine and seminal fluid samples as well as biopsies, organs, tissues or cell samples. Preferably, the sample is selected from the group consisting of skin biopsy, nervous tissue biopsy, serum and blood. More preferably, the sample is a skin biopsy. The sample may be treated prior to its use. In particular, skin biopsies may be treated to isolate fibroblasts cells. Fibroblasts may be isolated using any method known by the skilled person. For example, the dermal component of the skin may be cut into small pieces and treated over night with collagenase type 1 and dispase. After centrifugation and resuspension, cells may be seeded in culture flasks and cultured in fibroblasts proliferation medium containing, for example, Dulbecco's minimum essential medium, 10% fetal calf serum, penicillin and streptomycin.

As used herein, the term “dispersion descriptor” refers to a measure of dispersion denoted how stretched or squeezed is a distribution of values. Preferably, this descriptor is selected from the group consisting of the variance, the standard deviation, and an interquantile range. Preferably, the interquantile range is the interquartile range or the interdecile range. The interquartile range is the difference between the upper and lower quartiles, i.e. between the 25^{th} percentile (splits lowest 25% of data) and the 75^{th} percentile (splits highest 25% of data). The interdecile range is the difference between the 1^{st} and the 9^{th} deciles, i.e. between the 10^{th} percentile (splits lowest 10% of data) and the 90^{th} percentile (splits highest 90% of data). In a preferred embodiment, the dispersion descriptor is selected from the group consisting of the variance, the standard deviation and the interquartile range. Methods for calculating these values are commonly known by the skilled person.

The methods of the invention as disclosed below, may be in vivo, ex vivo or in vitro methods, preferably in vitro methods.

In a first aspect, the present invention concerns a method of screening for compounds useful in the treatment of AD, wherein the method comprises

a) contacting living cells obtained from a sample from a subject affected with Alzheimer disease with a test compound; and

b) measuring in said contacted cells, the values of the mitochondrial behaviour variables variables (i) to (iii):

(i) the average cell area, or a dispersion descriptor of the areas of cells (V5);

(ii) a variable selected from the group consisting of the frequency of mitochondria displaying an area between 70 and 100 μm² (V29), the total area of regions containing entwined mitochondria to the total cell area (V7), and the frequency of mitochondria displaying an area between 100 and 200 μm² (V30), and any combination thereof; and

(iii) the average cell area covered with mitochondria expressed as a percent of total cell area, or a dispersion descriptor of the areas of cells covered with mitochondria (V11).

Optionally, the values of additional mitochondrial behaviour variables may be measured.

In one embodiment, the method further comprises measuring the values of the mitochondrial behaviour variables

(iv) a dispersion descriptor of the moving speeds of mitochondria (V3), and/or

(v) the frequency of mitochondria displaying an area between 2.6 and 3.1 μm² (V19).

In a preferred embodiment, the method comprises measuring the values of the mitochondrial behaviour variables (i) to (v), preferably measuring the values of variables V5, V7, V11, V3 and V19.

In an embodiment, the method further comprises providing a sample from a subject affected with AD.

The subject affected with AD may be at any stage of the disease, in particular at the asymptomatic stage or at early stage of the disease progression. Preferably, the subject is a PAD subject.

The screening method may be conducted on the sample of a subject affected with AD in order to select a suitable therapy, i.e. personalized medicine, or on a population of AD or PAD samples, e.g. for drug development or drug repositioning.

Preferably, the sample is selected from the group consisting of skin biopsy, nervous tissue biopsy, serum and blood.

According to the nature of the sample, living cells may be selected from the group consisting of fibroblasts, lymphocytes and neuronal cells directly obtained from the sample or from primary cultures of cells from said sample. Living cells may also be induced pluripotent stem cells derived from adult somatic cells, in particular from fibroblasts obtained from the sample. Preferably, cells are non-transformed living cells to obtain results as close as possible of the in vivo situation.

In a preferred embodiment, the sample is a skin biopsy and cells are fibroblasts. In particular, the skin biopsy may be treated in order to isolate or enriched the culture in fibroblasts.

In an embodiment, the method further comprises, before measuring the values of mitochondrial behaviour variables, labelling mitochondria contained in living cells obtained from the sample. Preferably mitochondria are labelled before step a).

Mitochondria may be labeled using any method commonly known by the skilled person. Preferably, the label is a fluorescent, luminescent or colored label. More preferably, the label is a fluorescent label. Mitochondria may be labelled using a probe specific of said organelle and/or by transfection of a reporter gene (e.g. a GFP-expressing construct with mitochondrion-targeted expression) and/or by microinjection inside live cells of a marker or dye specifically taken up by said organelle. All these techniques are well known by the man skilled in the art and some commercial kits are available for this type of labelling and should be used according to manufacturer's recommendations. In particular, mitochondria may be labelled using calcein and cobalt (Petronilli et al., 1998), fluorescent rhodamine derivatives such as Rhodamine 123, tetramethylrhodamine methyl ester (TMRM) and tetramethylrhodamine ethyl ester (TMRE), carbocyanine dyes, 10-N-Nonyl acridine orange (NAO) or a MitoTracker dye, in particular MitoTracker Green (MTG) or MitoTracker red (CMXRos). Preferably, mitochondria are labelled using a dye which is not sensitive to mitochondrial membrane potential. In particular, the dye can be selected from the group consisting of MitoTracker Green (MTG), carbocyanine dyes, 10-N-Nonyl acridine orange (NAO) and the combination calcein-cobalt. In a preferred embodiment, mitochondria are labelled using MitoTracker Green.

In another embodiment, mitochondria are not labelled and the values of the mitochondrial behaviour variables are measured using a label-free microscopic technique such as differential interference contrast (DIC) microscopy.

In step a), living cells are contacted with a test compound.

The test compound may be selected from the group consisting of chemical compounds, biological compounds, radiations, and any combination thereof.

In an embodiment, the test compound is a radiation, in particular a radiation selected from the group consisting of X-rays, gamma rays, alpha particles, beta particles, photons, electrons, neutrons, radioisotopes, and other forms of ionizing radiation, and any combination thereof.

In another embodiment, the test compound is a chemical compound, i.e. an organic or inorganic compound. For example, the test compound may be a drug authorized to be marketed for another application than AD, a compound from a high-throughput chemical library or a nucleic acid construct suitable for gene therapy. If the test compound is a drug authorized to be marketed for another application than AD, the method may be used for drug repositioning.

In a further embodiment, the test compound is a biological compound. The biological compound may be selected from the group consisting of proteins, lipids, nucleic acids, carbohydrates and any other biological molecules or complexes. It may also be a therapeutic cell used for cell therapy or engineered virus for virotherapy. In particular, therapeutic cells may be stem cells, progenitor cells, mature and functional cells for cell replacement therapy or genetically modified cells for cell-based gene therapy.

The technique for contacting living cells with the test compound may vary according to the nature of said compound and may be easily chosen by the skilled person. In particular, if the test compound is a chemical or biological compound, it may be added to the cell culture medium. For cell therapy, living cells obtained from the sample and therapeutic or genetically modified cells may be contacted using a co-culture system allowing or not direct contact between the cells. If the test compound is radiation, cells in culture medium may be submitted to radiation. If the test compound is a nucleic acid construct, it may be added to the culture medium in a suitable vehicle such as liposome, transfected or directly injected into the cells. All these techniques are well known by the skilled person.

In step b) of the method, the values of several mitochondrial behavior variables are measured in cells contacted with the test compound in step a). As shown in the experimental section, these variables have been selected by the inventors to constitute an AD-signature that is sufficient to segregate cells from AD patients to cells from healthy patients and that can be reversed when cells are contacted with a test compound useful in the treatment or prevention of AD.

The values of mitochondrial behavior variables are measured by analyzing images of mitochondria, preferably labeled mitochondria, observed in living cells. Images were taken, for example, at least every 10 s at high scan speed for at least 2 min, preferably every 0.1 to 10 s at high scan speed for at least 2 to 6 min. The image capture may be carried out using any suitable microscopic technique such as fluorescent microscope or differential interference-contrast (DIC) microscope, coupled to an image acquisition system. In particular, if mitochondria are labeled, the image capture may be carried out using a fluorescent microscope. If mitochondria are not labeled, the image capture may be carried out using a DIC microscope. The image acquisition device may be any device allowing capture of high-resolution frames at high speed such as a Charge-Coupled Device (CCD). In a preferred embodiment, the image capture is performed in three spatial dimensions. The image capture may be thus carried out using a microscope equipped with a motorized plate allowing the visualization of a sample in three dimensions. The measurements may be conducted in presence or after exposition to the test compound, preferably in presence of the test compound. Preferably, cells are kept at 37° C. during the image capture.

The first variable V5 relates to the general structure of cells and is the average cell area, or a dispersion descriptor of the areas of cells. The variable may be obtained by determining the area of each cell and calculating the average area from data of all observed cells. The variable may also be obtained by determining the area of each cell and calculating a dispersion descriptor of the areas of cells. Preferably the descriptor selected from the group consisting of the variance, the standard deviation and an interquantile range, more preferably selected from the group consisting of the variance, the standard deviation and the interquartile range. Preferably, the area of each cell is determined using an image analysis software.

The second variable is selected from the group consisting of V7, V29 and V30, and any combination thereof, wherein V29 is the frequency of mitochondria displaying an area between 70 and 100 μm², V7 is the total area of regions containing entwined mitochondria to the total cell area, and V30 is the frequency of mitochondria displaying an area between 100 and 200 μm².

The variables V29 and V30 relate to the frequency of mitochondria displaying a specific range of area. Preferably, the area of each mitochondrion is determined using image analysis software. The variables V29 and V30 are thus obtained by measuring the area of mitochondria and determining the number of mitochondria displaying an area between 70 and 100 μm² and 100 and 200 μm², respectively. The results are then expressed in percent of the total number of mitochondria.

The variable V7 is the total area of regions containing tangled mitochondria, i.e. mitochondria that are interlaced to a point where individualisation of single mitochondria is impossible, to the total cell area. This variable assesses the concordance between the directionality of mitochondria with that of the cytoskeleton and is correlated to the state of the relationship between mitochondria and the cytoskeleton.

The third variable, V11, is the average cell area covered with mitochondria expressed as a percent of total cell area, or a dispersion descriptor of the areas of cells covered with mitochondria. The variable may be obtained by determining, for each cell, the area covered with mitochondria and calculating the average area from data of all observed cells. The variable may also be obtained by determining, for each cell, the area covered with mitochondria and calculating a dispersion descriptor of the cell areas covered with mitochondria. Preferably the descriptor selected from the group consisting of the variance, the standard deviation and an interquantile range, more preferably selected from the group consisting of the variance, the standard deviation and the interquartile range. Preferably, the area covered with mitochondria for each cell is determined using an image analysis software.

The fourth variable, V3, is a dispersion descriptor of the moving speeds of mitochondria. The moving speed of mitochondria is assessed by tracking each mitochondrion from frame to frame and recording the speeds of each mitochondrion. The variable V3 may be then obtained by calculating a dispersion descriptor of the moving speeds of mitochondria. Preferably the descriptor selected from the group consisting of the variance, the standard deviation and an interquantile range, more preferably selected from the group consisting of the variance, the standard deviation and the interquartile range.

The fifth variable, V19, is the frequency of mitochondria displaying an area between 2.6 and 3.1 μm². The area of each mitochondrion may be determined using image analysis software. The variable V19 may be thus obtained by measuring the area of mitochondria and determining the number of mitochondria displaying an area between 2.6 and 3.1 μm². The results are then expressed in percent of the total number of mitochondria.

The 6^{th} variable, V12, is the average number of individual mitochondria per unit of cell area, or a dispersion descriptor of the numbers of individual mitochondria per unit of cell area. This number is preferably determined using an image analysis software by counting each mitochondrion on an image of the cell and expressing the result in number per unit of cell area, preferably per μm² of cell area. The variable may be obtained by calculating the average number, of mitochondria per unit of cell from several measurements. This variable may also be obtained by calculating a dispersion descriptor, preferably selected from the group consisting of the variance, the standard deviation and an interquantile range, more preferably selected from the group consisting of the variance, the standard deviation and the interquartile range.

The 7^{th} variable is selected from the group consisting of V1 and V34, and a combination thereof, wherein V1 is the average frequency of stops during trajectories of individual mitochondria and V34 is the average frequency of burst during displacement of individual mitochondria.

The variable V1 is the average frequency of stops during trajectories of individual mitochondria. This frequency is assessed by tracking each mitochondrion from frame to frame and recording the number of stops, i.e. the number of periods during which the mitochondrion remains immobile between two frames. The variable V1 is thus obtained by measuring the number of stops per unit time for each traced mitochondrion and calculating the average frequency of stops from data of all traced mitochondria.

The variable V34 is the average frequency of burst during displacement of individual mitochondria. This frequency is assessed by tracking each mitochondrion from frame to frame and recording the number of burst, i.e. the number of sudden displacements within 30% of the maximal displacements of the mitochondrion during the period of capture. The variable V34 is thus obtained by measuring the number of burst per unit time for each traced mitochondrion and calculating the average frequency of burst from data of all traced mitochondria.

In a preferred embodiment, a recording and data management device, e.g. a computer with a suitable software, is used to record and analyze images of mitochondria observed through the microscope.

The number of mitochondria and cells to be analyzed for each variable is easily determined by the skilled person using statistic methods. Preferably, at least 50, 80 or 100 mitochondria are analyzed for each variable, preferably from at least 3, 10 or 15 cells. In a particular embodiment, at least 50, 80 or 100 mitochondria are analyzed for variables V1, V3 and V34 and at least 100, 250, 500, 800, 900 or 1000 mitochondria are analyzed for variables V5, V7, V11, V12, V19, V29 and V30.

The method may comprise measuring the values of a combination of variables selected from the group consisting of V5, V7 and V11; V5, V29 and V11; V5, V30 and V11; V5, V7, V11 and V3; V5, V29, V11 and V3; V5, V30, V11 and V3; V11; V5, V7, V11 and V19; V5, V29, V11 and V19; V5, V30, V11 and V19; V11; V5, V7, V11, V3 and V19; V5, V29, V11, V3 and V19; V5, V30, V11, V3 and V19; V11; V5, V7, V11, V3, V19 and V12; V5, V29, V11, V3, V19 and V12; V5, V30, V11, V3, V19 and V12; V11; V5, V7, V11, V3, V19 and V1; V5, V29, V11, V3, V19 and V1; V5, V30, V11, V3, V19 and V1; V11; V5, V7, V11, V3, V19 and V34; V5, V29, V11, V3, V19 and V34; V5, V30, V11, V3, V19 and V34; V11; V5, V7, V11, V3, V19, V12 and V1; V5, V29, V11, V3, V19, V12 and V1; V5, V30, V11, V3, V19, V12 and V1; V11; V5, V7, V11, V3, V19, V12 and V34; V5, V29, V11, V3, V19, V12 and V34; V5, V30, V11, V3, V19, V12 and V34, wherein V5, V7, V29, V30, V11, V3, V19, V12, V1 and V34 are as defined above.

In a particular embodiment, the method comprises measuring the variables V5, V7 and V11, as defined above. In a preferred embodiment, the method comprises measuring the variables V5, V7, V11, V3 and V19 as defined above.

Alternatively, the variables can be measured on samples from a population of AD or PAD patients. The values obtained for each variable are then averaged.

Each variable may be weighted in order to adjust their importance.

The method of screening of the invention may further comprise comparing the values of the variables obtained in step b) in presence of the test compound with the values obtained in absence of the test compound. Preferably, the values in absence of the test compound are obtained on cells from the same sample than in step b) before contacting the test compound. In a particular embodiment, these values are obtained after labelling mitochondria and before step a), i.e. on cells with labelled mitochondria before contacting the test compound. The value of each variable is measured as detailed above. Values obtained without the test compound may be used as negative control to identify compounds that could be useful in the treatment of AD.

The method may also further comprise comparing the values of variables obtained in presence, and optionally in absence of the test compound, with the values of said variables measured in a sample obtained from a healthy subject (in absence of the test compound). Preferably, the healthy subject is about the same age as the AD patient providing the AD sample. The value of each variable is measured as detailed above. Values obtained from the sample from the healthy subject may be used as positive control to identify compounds that could be useful in the treatment of AD. Alternatively, this positive control can be obtained by measuring the variables on samples from a population of healthy subject. The values obtained for each variable are then averaged.

In a particular embodiment, the values of variables of the AD signature are measured in presence of several concentrations of the test compound in order to determine the dose-response effect of the test compound on AD.

The significance of differences of measured values may be determined using any suitable statistic test such as ANOVA.

Using a discriminating equation, the values obtained for the variables for each dose of the test compound, may be represented as a score. The effect of each dose may be evaluated in respect to the score obtained for the negative and/or positive controls.

In particular, the score for each variable may be calculated using the following ratio:

Score of the variable Vx=(NC−Var)*100/(NC−PC)

(NC: value or average value of the negative control; PC: value or average value of the positive control; Var: value of the variable).

A global percentage of phenotypic rescue may be obtained by adding up the scores of each measured variable.

The results may thus be expressed as a percentage of phenotypic rescue, the positive control (healthy sample) being 100% and the negative control (AD sample in absence of the test compound) being 0%.

Test compound providing a positive phenotypic rescue, i.e. a compound that is able to partially or totally reverse the AD signature, is identified as potentially useful in the treatment of AD. In a particular embodiment, a test compound is identified as potentially useful if the phenotypic rescue is above 50%, more preferably above 60%, 70%, 80% or 90%.

Preferably, a test compound is identified as potentially useful in the treatment of AD if all measured variables have a positive score, i.e. if a rescue is observed for each variable.

In another aspect, the present invention concerns a method for diagnosing Alzheimer disease in a subject, wherein the method comprises measuring in living cells obtained from a sample from said subject the values of the mitochondrial behaviour variables (i) to (iii):

(i) the average cell area, or a dispersion descriptor of the areas of cells (V5);

(ii) a variable selected from the group consisting of the frequency of mitochondria displaying an area between 70 and 100 μm² (V29), the total area of regions containing entwined mitochondria to the total cell area (V7), and the frequency of mitochondria displaying an area between 100 and 200 μm² (V30), and any combination thereof; and

(iii) the average cell area covered with mitochondria expressed as a percent of total cell area, or a dispersion descriptor of the areas of cells covered with mitochondria (V11).

Optionally, the method may further comprise measuring the values of the mitochondrial behaviour variables (iv) and/or (v):

(iv) a dispersion descriptor of the moving speeds of mitochondria (V3), and

(v) the frequency of mitochondria displaying an area between 2.6 and 3.1 μm² (V19).

All embodiments described above for the method of screening are also contemplated in this aspect.

The method may further comprise providing a sample from the subject to be diagnosed.

Preferably, mitochondria contained in living cells are labelled before measuring mitochondrial behavior variables.

The subject may have clinical signs that resemble AD or may be without any symptom.

The method may further comprise conducting tests, in particular genetic tests, to determine if the subject is at risk of developing AD. For example, it is known that the APOEε4 allele, mutations in the amyloid peptide precursor, TREM2, PSEN1 or PSEN2 gene increases the risk of the disease. Other parameters such as age, gender, cardio-vascular risks, diabetes, depression or prior occurrence of brain trauma, can also be taken into account to evaluate the risk of developing AD.

The method may further comprise determining if said subject is affected with Alzheimer disease based on the measured values of mitochondrial behaviour variables. The diagnosis of AD may be obtained by comparing the score obtained with the sample of the subject with the score of the sample obtained from a healthy patient or from AD patient.

In a particular embodiment, the method comprises measuring the mitochondrial behaviour variables V5, the 2^{nd} variable, i.e. V7, V29 and/or V30, preferably V7, and V11 as defined above, a gain of function in the 2^{nd} variable, i.e. V7, V29 and/or V30, preferably V7, and V11 and a loss of function in variable V5, by comparison with the values of these variables obtained from a healthy sample, is indicative of AD. The method may further comprise calculating the z-scores of measured variables. In particular, in AD sample the z-scores of the 2^{nd} variable, i.e. V7, V29 and/or V30, preferably V7, and V11 are positive and the z-scores of variable V5 is negative. Furthermore, V3 and/or V19 variables may be used to segregate AD and PAD patients. Thus, in another particular embodiment, the method comprises measuring the mitochondrial behaviour variables V5, the 2^{nd} variable, i.e. V7, V29 and/or V30, preferably V7, V11, and V3 and/or V19 as defined above, preferably measuring the variables V5, V7, V11 and V19.

In this embodiment, a gain of function in the 2^{nd} variable, i.e. V7, V29 and/or V30, preferably V7, and V11 and a loss of function in variables V5 and V19, by comparison with the values of these variables obtained from a healthy sample, is indicative of AD. Alternatively, a gain of function in the 2^{nd} variable, i.e. V7, V29 and/or V30, preferably V7, V11 and V19, and optionally V3, and a loss of function in variable V5, by comparison with the values of these variables obtained from a healthy sample, is indicative of pre-symptomatic AD (PAD).

The present invention also concerns a method for providing useful information for the diagnosis of Alzheimer disease in a subject, wherein the method comprises measuring in living cells obtained from a sample from said subject the values of the mitochondrial behaviour variables (i) to (iii):

(i) the average cell area, or a dispersion descriptor of the areas of cells (V5);

(ii) a variable selected from the group consisting of the frequency of mitochondria displaying an area between 70 and 100 μm² (V29), the total area of regions containing entwined mitochondria to the total cell area (V7), and the frequency of mitochondria displaying an area between 100 and 200 μm² (V30), and any combination thereof; and

(iii) the average cell area covered with mitochondria expressed as a percent of total cell area, or a dispersion descriptor of the areas of cells covered with mitochondria (V11).

Optionally, the method may further comprise measuring the values of the mitochondrial behaviour variables (iv) and/or (v):

(iv) a dispersion descriptor of the moving speeds of mitochondria (V3), and

(v) the frequency of mitochondria displaying an area between 2.6 and 3.1 μm² (V19).

All embodiments described above are also contemplated in this aspect.

In another aspect, the present invention also concerns a method for monitoring the response of a subject affected with Alzheimer disease to therapy, or for selecting a subject affected with Alzheimer disease for therapy, wherein the method comprises

a) measuring in living cells obtained from a sample from said subject, before and after the administration of the treatment, the values of the mitochondrial behaviour variables (i) to (iii):

(i) the average cell area, or a dispersion descriptor of the areas of cells (V5);

(ii) a variable selected from the group consisting of the frequency of mitochondria displaying an area between 70 and 100 μm² (V29), the total area of regions containing entwined mitochondria to the total cell area (V7), and the frequency of mitochondria displaying an area between 100 and 200 μm² (V30), and any combination thereof; and

(iii) the average cell area covered with mitochondria expressed as a percent of total cell area, or a dispersion descriptor of the areas of cells covered with mitochondria (V11),

and, optionally, the values of the mitochondrial behaviour variables (iv) and/or (v):

(iv) a dispersion descriptor of the moving speeds of mitochondria (V3), and

(v) the frequency of mitochondria displaying an area between 2.6 and 3.1 μm² (V19); and

b) comparing the measured values obtained before and after the administration of the treatment in step a).

All embodiments described above for the method of screening are also contemplated in this aspect.

The subject affected with AD may be at any stage of the disease, in particular at the asymptomatic or symptomatic stage. Preferably, the subject is at early stage of the disease progression. More preferably, the subject is a PAD subject.

Preferably, mitochondria contained in living cells are labelled before measuring mitochondrial behavior variables.

The method may further comprise providing a sample from the subject before and/or after the administration of the treatment, preferably before and after the treatment.

The therapy may comprise administering one or several chemical or biological compounds, as well as radiations, as defined above.

As explained above for the method of screening, the values obtained for the mitochondrial behaviour variables before and after the administration of the treatment may be represented as a score. The effect of the treatment may be thus evaluated by comparing the scores obtained before and after the treatment. Optionally, the scores may also be compared with the score obtained from a healthy sample or from a population of healthy samples.

In a preferred embodiment, a score is calculated for each variable using the following equation: score=(NC−Var)*100/(NC−PC), wherein NC is the value obtained before the administration of the treatment, PC is the value or average value obtained with healthy sample(s), and Var is the measured value of the variable. The patient is responsive to the therapy or is susceptible to benefit from the therapy when all measured variables have a positive score. If one or several variables have negative scores, the therapy may worsen the symptoms of the disease and should be stopped or avoided.

The results may also be expressed as a percentage of phenotypic rescue, the positive control (healthy sample) being 100% and the negative control (AD or PAD sample, preferably AD sample, without any treatment, i.e. the AD sample obtained before the administration of the therapy) being 0%. A positive phenotypic rescue is indicative that the AD or PAD patient is responsive to the therapy or is susceptible to benefit from the therapy. On the contrary, a negative phenotypic rescue indicates that the therapy may worsen the symptoms of the disease and should be stopped or avoided.

Further aspects and advantages of the present invention will be described in the following examples, which should be regarded as illustrative and not limiting.

### EXAMPLES

### Example 1

### Alzheimer Disease Signature

Skin Biopsy Sample Collection

Skin biopsies were collected from 18 human donors comprising 5 healthy subjects, 8 Ad patients and 4 pre-symptomatic AD patients (Table 1).

TABLE 1
Cohort characteristics

| | | | Pre-symptomatic |
|---|---|---|---|
| | | Alzheimer Disease | Alzheimer Disease |
| Descriptive | Healthy | (AD) | (PAD) |
| N = 17 | 5 | 8 | 4 |
| Age | 40.2 +/− 6.45 | 46.5 +/− 4.96 | 36.67 +/− 5.13 |
| Gender | 66.6% female | 50% female | 40% female |
| | 33.3% male | 50% male | 60% male |
| Duration | N/A | 4.17 +/− 3.0 | N/A |
| Ethnicity | 100% | 87.5% caucasian | 100% caucasian |
| | caucasian | 12.5% asian | |
| Confounding | 1 subject with | 1 patient is also | Group of |
| factors | untreated mild | affected by a brain | presymptomatic |
| | depression, | tumor | patients at different |
| | 1 subject | 2 patients carrier of | stages (At risk, MCI |
| | asymptomatic | APOE4 allelle | or PSEN1 mutation |
| | carrier of | 2 patients had AD | carrier) |
| | wolfram | confirmed by brain | |
| | syndrome | autopsy post- | |
| | | mortem | |

The cohort was chosen to minimize possible confounding age effects by limiting the age range within roughly a decade around 40 years old respectively in healthy, pre-symptomatic and diseased subjects. Pre-symptomatic patients were chosen to find a signature that can discriminate the two groups AD and PAD, and to have the possibility to diagnose Alzheimer disease at early stage. The PAD group includes two patients with mild cognitive impairment (MCI), one patient with presenilin mutation and one patient at risk of developing the disease who evolved into the disease later. AD was confirmed through post-mortem biopsies in 25% of the AD group. Another 25% expressed the ApoE4 genetic allele associated with higher risk of developing the disease.

Cell Culture

Patient-derived fibroblasts were cultured in DMEM containing 15% fetal calf serum. Cells of the sample were expanded and found stable for at least 16-20 passages.

Dynamical Imaging

Fibroblasts were labelled with MitoTracker green, a cell-permeant mitochondrial dye not sensitive to mitochondrial membrane potential, for 30 minutes. Images were recorded from an epifluorescence microscope continuously for 6 minutes. Cells were kept at 37° C. for the duration of the image capture. For each experimental condition, 3 to 15 cells were recorded per well from three independent plates, i.e. up to 6000 individual mitochondria. The maximum duration for data acquisition was 30 minutes (i.e. five cells observed during 6 min). Images were captured with a Zeiss Axioplan II microscope along three dimensions in space and in time.

Identification of Markers of the AD Signature

37 variables defining mitochondrial behaviour and labelled V1 to V37 were simultaneously measured. These variables reflected the mitochondrial motility, the mitochondrial morphology, the mitochondrial reticular network or relationship with the cytoskeleton and the mitochondrial permeability.

Using statistical methods (Principal Component Analysis, Hierarchical cluster classification, Discriminant Analysis and ANOVA), three variables (V5, V7 and V11) were found to be sufficient to distinguish healthy from AD and PAD samples with 100% accuracy.

V5: the average cell area, or a dispersion descriptor of the areas of cells;

V7: the total area of regions containing entwined mitochondria to the total cell area;

V11: the average cell area covered with mitochondria expressed as a percent of total cell area, or a dispersion descriptor of the areas of cells covered with mitochondria.

The inventors found that, in this signature, V7 could be replaced by

V29: the frequency of mitochondria displaying an area between 70 and 100 μm²; or

V30: the frequency of mitochondria displaying an area between 100 and 200 μm².

Furthermore, using two additional variables, i.e. V3 and V19, it was also found that it was possible to properly classify 100% of the subjects in the three groups of interest (healthy, AD and PAD groups).

V3: a dispersion descriptor of the moving speeds of mitochondria; and

V19: the frequency of mitochondria displaying an area between 2.6 and 3.1 μm².

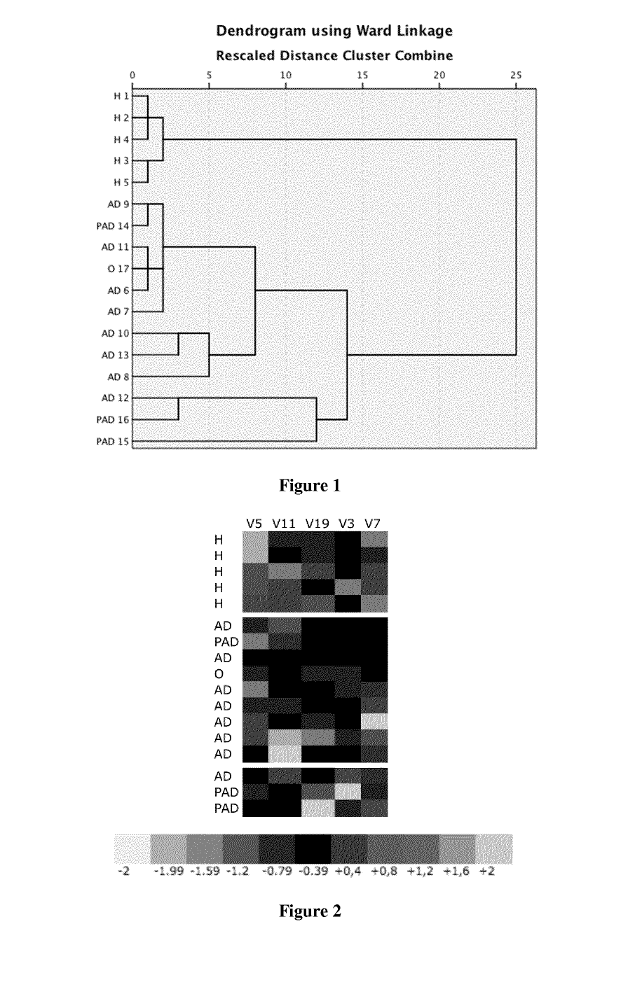

The dendogram of the hierarchical cluster analysis applying the Ward's method and obtained with the five variables (V3, V5, V7, V11 and V19) is shown in FIG. 1. The cluster analysis shows three subgroups composed of (i) healthy subjects (top group), (ii) AD patients in which subject PAD 14 is misplaced (middle group), and (iii) PAD patients in which subject AD 12 is misplaced (bottom group). Misplaced PAD and AD patients have singularities that may explain their classification in improper groups. The PAD subject #14 bears a mutation in the presenilin gene 1, while AD patient #12 is of Asiatic ethnicity compared to Caucasian ethnicity in all other subjects.

For each patient of the cohort, the number of mitochondria studied for each variable was from 3315 to 15237 for V5, from 139 to 461 for V3, from 3315 to 15237 for V7, from 3315 to 15237 for V11 and from 3315 to 15237 for V19. From these results, a heat map corresponding to the actual values obtained in the subjects was derived (FIG. 2).

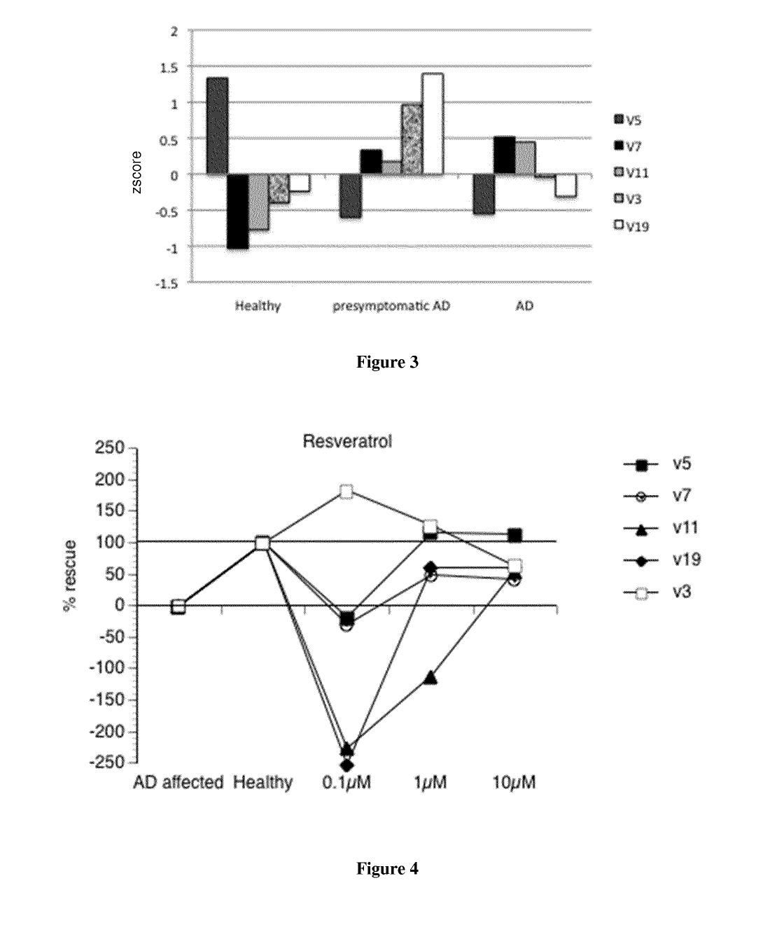

AD patients are characterized by a loss of function in variables V5 and a gain of function in V7 and V11. PAD subjects are characterized by a loss of function in V5 and a gain of function in V3, V7, V11 and mostly V19 (FIG. 3).

The robustness of the signature comprising the five variables was tested and confirmed by analyzing about 39,000 additional mitochondria from 215 cells in 9 independent experiments and repeated the hierarchical cluster analysis using a chosen donor pair (one healthy subject and one AD patient). This donor pair was subsequently used in example 2.

### Example 2

### Reversal of the AD-Signature

Reference Compounds

Resveratrol is a plant polyphenol found in grapes and red wine. Resveratrol is associated with beneficial effects on aging, metabolic disorders, inflammation and cancer. Despite poor bioavailability compromised by its physicochemical properties, resveratrol was shown to promote the non-amyloidogenic cleavage of the amyloid precursor protein, enhance clearance of amyloid beta-peptides, and reduce neuronal damage (Li et al., 2012; Vang et al., 2011; Smoliga et al., 2011; Patel et al., 2011; Timmers et al., 2011; Albani et al 2010). A clinical trial is currently underway to test the therapeutical potential of resveratrol in Alzheimer disease in the US.

Allopregnanolone is an agonist of GABAA receptor and is capable of stimulating endogenous neurogenesis in neocortical areas of AD patients (Brinton et al., 2006). Conflicting results exist in the literature about the therapeutic effects of allopregnanolone treatment in various transgenic mouse models of AD (Chen et al., 2011; Singh et al., 2012; Bengtsson et al., 2012; Bengtsson et al., 2013). Allopregnanolone is currently being tested in clinical trials.

Imatinib was also used as it was reported to have activity on both amyloid fibrillation (Fraering et al., 2005; Sutcliffe et al., 2011) and Tau phosphorylation (Cancino et al., 2008). However, Imatinib does not cross the brain-blood barrier and is associated with significant cardiac toxicity.

Materials and Methods

Patient-derived fibroblasts were cultured in DMEM containing 15% fetal calf serum and 0.5% DMSO, a concentration known to be inert on the mitochondrial behaviour.

Cells were incubated with different doses of reference compounds (resveratrol, allopregnanolone or imatinib) diluted in DMSO or with the vehicle only (DMSO) for 30 minutes and observed through time-lapse video-microscopy for another 30 minutes during dynamic image capture.

Reference compounds were tested at 3 or 4 doses spanning 3.5 log (10, 1, 0.1 and 0.05 μM). These concentrations are consistent with circulating levels of most drugable small molecules. They are within the range of doses centered on cMax when PK/PD (Pharmacokinetic/Pharmacodynamic) studies are available.

The negative control consists in affected cells treated with the vehicle only (DMSO), the positive control consists in healthy cells treated with the vehicle only (DMSO).

Reference compounds were tested on affected cells and compared to the 2 controls.

All raw data were expressed as a percent of control values (Healthy control=100%, AD control=0%).

Data analysis of the signature variables was particularly tuned to identify bifurcation in the logic of the descriptor measured. These bifurcations and the type of adaptation put in place by the mitochondrial reticular network give us significant insights on how the cell adapts to the test element. Impact of the disease on each individual variable was analyzed through a classical linear analysis with an analysis of variance (ANOVAs). Significant differences between groups are sought through a Bonferroni post-hoc analysis.

Results

Resveratrol

Dose-response analysis of the effect of Resveratrol on the 5 variables constituting the AD-signature is shown in FIG. 4.

Effects are expressed as a percent of the diseased phenotype. 0% represents the diseased status while 100% represents the healthy status.

Resveratrol was tested at 0.1, 1 and 10 μM in DMSO. It was efficient in reversing the disease phenotype for V3 but showed seesaw effects for V5 and V7. The effects of resveratrol greatly worsened the disease-phenotype for V11 and V19.

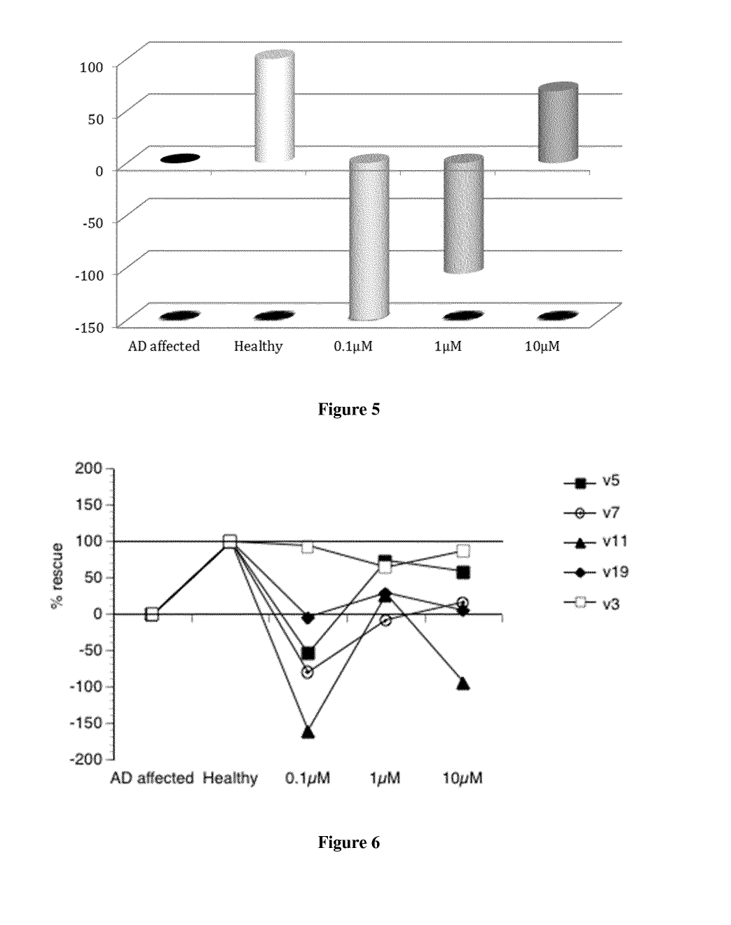

To express the weighted effect of the different variable on the overall rescue, a discriminant equation was applied. The resulting activity profile (FIG. 5) shows that 0.1 and 1 μM resveratrol did not provide rescue to the AD phenotype but a detrimental effect with worsening of the diseased status. On the contrary, at 10 μM, resveratrol provided 63% recovery.

Allopregnanolone

Dose-response analysis of the effect of allopregnanolone on the 5 variables constituting the AD-signature is shown in FIG. 6.

Effects are expressed as a percent of the diseased phenotype. 0% represents the diseased status while 100% represents the healthy status.

Allopregnanolone was tested at 0.1, 1 and 10 μM in DMSO. A rescue was obtained at all tested doses on V3 and at 1 and 10 μM on V5. There were no effect of allopregnanolone on V19. On V7 and V11, effects of allopregnanolone were detrimental at the lowest tested dose and tended to improve with increasing concentration of the drug but not to a level where the disease phenotype was reversed.

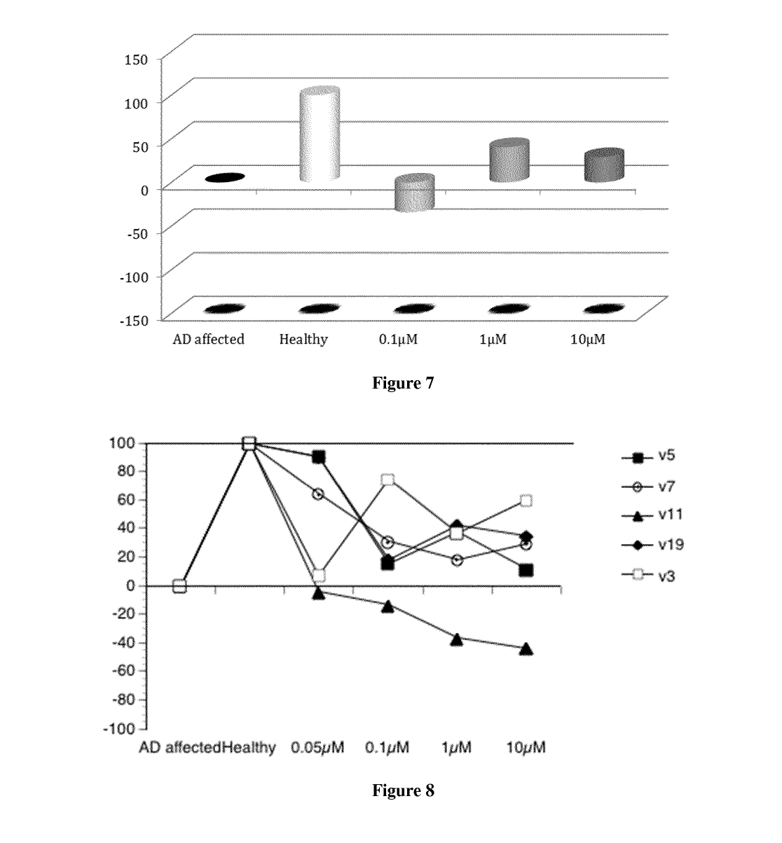

To express the weighted effect of the different variable on the overall rescue, a discriminant equation was applied. The resulting activity profile (FIG. 7) shows that 1 and 10 μM allopregnanolone provided a dose-dependent rescue to the AD phenotype culminating at 1 μM with an overall beneficial effect of 36%.

Imatinib

Dose-response analysis of the effect of imatinib on the 5 variables constituting the AD-signature is shown in FIG. 8.

Effects are expressed as a percent of the diseased phenotype. 0% represents the diseased status while 100% represents the healthy status.

Imatinib was tested at 0.05, 0.1, 1 and 10 μM in DMSO. It showed a significant disease-modifying capability at the lowest tested dose with modulation of V5, V7 and V19 to a level comparable to that of healthy subject-derived cells. V3 and V11 were not affected at 0.05 μM. V3 tended to improve with increasing doses whereas V11 worsened.

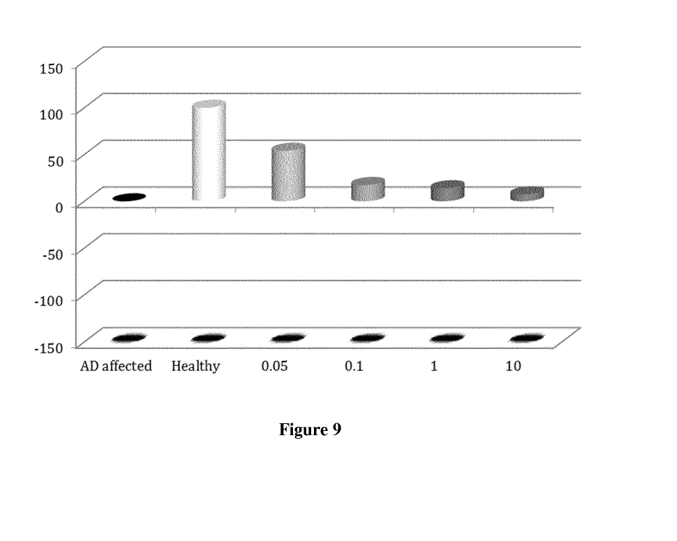

To express the weighted effect of the different variable on the overall rescue, a discriminant equation was applied. The resulting activity profile (FIG. 9) shows that imatinib showed a dose-dependent decrease in efficacy in the overall rescue of the diseased phenotype (FIG. 9). The highest rescue of 54% was seen at the lowest tested dose of 0.05 μM. Lower doses were tested to see if there were as effective but showed no effect (not shown) suggesting a very narrow window of opportunity for AD treatment with this drug.

### CONCLUSION

These results show that the AD signature established by the inventors and comprising the variables is sufficient to segregate AD, PAD and healthy patients and may constitute the basis for AD diagnosis at several stages of disease progression including pre-symptomatic stages from minimally invasive skin biopsies.

The inventors have also demonstrated that this AD signature can be reversed and thus allows the study of disease-modifying properties of compounds even in a dose-dependent manner. As example, they have shown that three compounds, resveratrol, allopregnanolone and imatinib, previously shown to have disease reversal capability in animals but that are not drugable in humans for physico-chemical properties and tolerance issues, have the ability to reverse the AD-signature in a dose-dependent manner. This signature can thus be used as a powerful tool to screen and identify novel drugs useful for AD treatment.

Because one can monitor the evolution of this signature prior or after administration of a compound in an AD patient, this signature can also be used as a surrogate marker for treatment efficacy, in particular in clinical trials.

### REFERENCES

Albani et al. J Alzheimers Dis. 2010; 19(1):11-26.
Bengtsson et al. Curr Alzheimer Res. 2013 January; 10(1):38-47.
Bengtsson et al. Journal of Alzheimer's Disease; 2012, 31(1): 71-84.
Brinton et al. Curr Alzheimer Res. 2006 July; 3(3):185-90.
Cancino et al. Brain. 2008 September; 131(Pt 9):2425-42.
Chen et al. PLoS One. 2011; 6(8):e24293.
Fraering et al. J Biol Chem. 2005 Dec. 23; 280(51):41987-96.
Li et al. Curr Pharm Des. 2012; 18(1):27-33.
Patel et al. Ann N Y Acad Sci. 2011 January; 1215:161-9.
Singh et al. Neurobiol Aging. 2012 August; 33(8):1493-506.
Smoliga et al. Mol Nutr Food Res. 2011 August; 55(8):1129-41.
Sutcliffe et al. J Neurosci Res. 2011 June; 89(6):808-14.
Timmers et al. Cell Metab. 2011 Nov. 2; 14(5):612-22.
Vang et al. PLoS One. 2011; 6(6):e19881.

1-22. (canceled)

23. An in vitro method of screening for compounds useful in the treatment of Alzheimer's disease, wherein the method comprises:
a) contacting living cells obtained from a sample from a subject affected with Alzheimer's disease with a test compound; and
b) measuring in said contacted cells, the values of the mitochondrial behaviour variables (i) to (iii):
(i) the average cell area, or a dispersion descriptor of the areas of cells (V5);
(ii) a variable selected from the group consisting of the frequency of mitochondria displaying an area between 70 and 100 μm² (V29), the total area of regions containing entwined mitochondria to the total cell area (V7), the frequency of mitochondria displaying an area between 100 and 200 μm² (V30), and any combination thereof; and
(iii) the average cell area covered with mitochondria expressed as a percent of total cell area, or a dispersion descriptor of the areas of cells covered with mitochondria (V11),
and, optionally, the values of the mitochondrial behaviour variables (iv) and/or (v):
(iv) a dispersion descriptor of the moving speeds of mitochondria (V3); and
(v) the frequency of mitochondria displaying an area between 2.6 and 3.1 μm² (V19).

24. The method according to claim 23, further comprising comparing the values obtained in step b) with values obtained in the absence of said test compound.

25. The method according to claim 23, further comprising calculating a score for each variable using the following equation:
score=(NC−Var)*100/(NC−PC),
wherein NC is the value or average value obtained with the sample(s) from subject(s) affected with Alzheimer's disease in the absence of the test compound, PC is the value or average value obtained with healthy sample(s), and Var is the measured value of the variable.

26. The method according to claim 25, wherein a test compound is identified as useful in the treatment of Alzheimer's disease when all measured variables have a positive score.

27. The method according to claim 23, wherein the method comprises measuring the mitochondrial behaviour variables V3, V5, V7, V11 and V19.

28. The method according to claim 23, wherein the method further comprises measuring at least one additional mitochondrial behaviour variable selected from the group consisting of variables (vi) and (vii):
(vi) the average number of individual mitochondria per unit of cell area, or a dispersion descriptor of the numbers of individual mitochondria per unit of cell area (V12); and
(vii) a variable selected from the group consisting of the average frequency of stops during trajectories of individual mitochondria (V1), the average frequency of burst during displacement of individual mitochondria (V34), and a combination thereof.

29. The method according to claim 23, wherein the method further comprises comparing the measured values of the mitochondrial behaviour variables with the values of said variables measured in a sample obtained from a healthy subject.

30. The method according to claim 23, wherein the sample is selected from the group consisting of a skin biopsy sample, nervous tissue biopsy sample and serum or blood sample.

31. The method according to claim 23, wherein the living cells are selected from the group consisting of fibroblasts, induced pluripotent stem cells derived from fibroblasts, lymphocytes and neuronal cells.

32. The method according to claim 31, wherein the living cells are fibroblasts.

33. The method according to claim 23, wherein the dispersion descriptor is selected from the group consisting of the variance, the standard deviation and an interquantile range.

34. The method according to claim 23, wherein the mitochondria contained in living cells are labeled before measuring the values of the mitochondrial behaviour variables.

35. The method according to claim 34, wherein the mitochondria in living cells are labeled with a fluorescent label.

36. The method according to claim 23, wherein the values of the mitochondrial behaviour variables are obtained from images captured using a fluorescence microscope or DIC microscope coupled to an image acquisition device.

37. An in vitro method for diagnosing Alzheimer's disease in a subject, wherein the method comprises measuring in living cells obtained from a sample from said subject the values of the mitochondrial behaviour variables (i) to (iii):
(i) the average cell area, or a dispersion descriptor of the areas of cells (V5);
(ii) a variable selected from the group consisting of the frequency of mitochondria displaying an area between 70 and 100 μm² (V29), the total area of regions containing entwined mitochondria to the total cell area (V7), the frequency of mitochondria displaying an area between 100 and 200 μm² (V30), and any combination thereof; and
(iii) the average cell area covered with mitochondria expressed as a percent of total cell area, or a dispersion descriptor of the areas of cells covered with mitochondria (V11), and optionally the values of the mitochondrial behaviour variables (iv) and/or (v):
(iv) a dispersion descriptor of the moving speeds of mitochondria (V3); and
(v) the frequency of mitochondria displaying an area between 2.6 and 3.1 μm² (V9).

38. The method according to claim 37, wherein the subject has clinical signs that resemble Alzheimer's disease or is without any symptom.

39. The method according to claim 37, further comprising calculating the z-scores of measured variables.

40. The method according to claim 39, wherein positive z-scores for the 2^{nd} variable and the variable V11 and negative z-score of variable V5 are indicative that the subject is affected with Alzheimer's disease.

41. The method according to claim 39, wherein positive z-scores for the 2^{nd} variable, V11 and V19 and/or V3, and negative z-score of variable V5, are indicative that the subject is affected with Alzheimer's disease at the asymptomatic stage.

42. An in vitro method for monitoring the response of a subject affected with Alzheimer's disease to therapy, wherein the method comprises:
a) measuring in living cells obtained from a sample from said subject, before and after the administration of the treatment, the values of the mitochondrial behaviour variables (i) to (iii):
(i) the average cell area, or a dispersion descriptor of the areas of cells (V5);
(ii) a variable selected from the group consisting of the frequency of mitochondria displaying an area between 70 and 100 μm² (V29), the total area of regions containing entwined mitochondria to the total cell area (V7), the frequency of mitochondria displaying an area between 100 and 200 μm² (V30), and any combination thereof; and
(iii) the average cell area covered with mitochondria expressed as a percent of total cell area, or a dispersion descriptor of the areas of cells covered with mitochondria (V11),
and, optionally, the values of the mitochondrial behaviour variables (iv) and/or (v):
(iv) a dispersion descriptor of the moving speeds of mitochondria (V3); and
(v) the frequency of mitochondria displaying an area between 2.6 and 3.1 μm² (V19); and
b) comparing the measured values obtained before and after the administration of the treatment in step a).

43. The method according to claim 42, further comprising calculating a score for each variable using the following equation:
score=(NC−Var)*100/(NC−PC),
wherein NC is the value obtained before the administration of the treatment, PC is the value or average value obtained with healthy sample(s), and Var is the measured value of the variable.

44. The method according to claim 43, wherein the patient is responsive to the therapy or is susceptible to benefit from the therapy when all measured variables have a positive score.
